(19) United States
(12) Patent Application Publication
(10) Pub. No.: US 2011/0004096 A1
(43) Pub. Date: Jan. 6, 2011

(54) ELECTROMAGNETIC CELLULAR TOMOGRAPHY

(75) Inventors: Chin-Lin GUO, Pasadena, CA (US); Hua Wang, Pasadena, CA (US)

Correspondence Address: MILSTEIN ZHANG & WU LLC, 49 LEXINGTON STREET, SUITE 6, NEWTON, MA, 02465-1062, US

(73) Assignee: California Institute of Technology, Pasadena, CA (US)

(21) Appl. No.: 12/830,975

(22) Filed: Jul. 6, 2010

Related U.S. Application Data
(60) Provisional application No. 61/223,341, filed on Jul. 6, 2009.

(51) Int. Cl. A61B 5/05

(52) U.S. Cl. 600/425

(57) ABSTRACT

The invention provides an electromagnetic cellular tomograph and methods of operating such a device. An array of structures is configured to apply probe signals to cells or tissues of interest that are held in a sample holder. The array also includes structures that can receive a response signal from the sample of interest. Data processing and control circuits are provided to manipulate and analyze the response and to allow the result to be recorded, transmitted to other data systems, or displayed to a user.

### CROSS-REFERENCE TO RELATED APPLICATIONS

This application claims priority to and the benefit of co-pending U.S. provisional patent application Ser. No. 61/223,341 filed Jul. 6, 2009, which application is incorporated herein by reference in its entirety. This application is also related to U.S. patent application Ser. No. 12/399,603 filed Mar. 6, 2009 and published as US 2009-0267596 A1, U.S. patent application Ser. No. 12/559,517 filed Sep. 15, 2009 and published as US 2010-0134097 A1, U.S. patent application Ser. No. 12/710,334 filed Feb. 22, 2010, U.S. patent application Ser. No. 12/713,128 filed Feb. 25, 2010, and H. Wang and A. Hajimiri, “Design of Inductors with Uniform Magnetic Field Strength in the Near-Field,” U.S. Provisional patent application Attorney Docket No. CIT-5505-P filed Dec. 23, 2009, each of which applications is incorporated herein by reference in its entirety.

### FIELD OF THE INVENTION

The invention relates to bioanalytical systems and methods in general and particularly to a tomographic system and method that can analyze individual cells and tissues.

### BACKGROUND OF THE INVENTION

Non-optical methods for bioassay have attracted attention in the interdisciplinary field of biology, applied physics and microelectronics. In particular, superparamagnetic micro/nano beads have been extensively studied as a promising candidate for cell/bio-molecule sensing since their magnetic behavior can be detected without using any expensive imaging systems. In addition, there are other advantages for magnetic-bead-based sensors. First, no bio-system can generate comparable magnetic signals, which provides a relative quite sensing background. Moreover, unlike currently dominant fluorescent labels, magnetic beads do not have signal quenching or decaying problems, because their signal has a time-invariant relationship with respect to external excitation, which is favorable for signal to noise ratio (SNR) improvement by signal averaging. Finally, magnetic beads have been shown to have the possibility of manipulating attached cells/macromolecules. This can be used for bio-sample delivery, concentration/separation and affinity binding facilitation without using valve/channel based conventional micro-fluidic systems.

However, sensing magnetic micro/nano particles remains as a challenging task. This is because their superparamagnetic property only offers a low effective relative permeability value (μᵣ), normally around 2 to 3, which leads to small magnetic signals. In addition, since traditional toroid shaped particles cannot be adopted to planar sensors, conventional magnetic excitation and sensing has to be carried out in an open-magnetic-loop fashion, where demagnetization effect arises and further degrades the sensitivity. Various detection methods have been proposed to address this sensing challenge. Traditionally, superconducting quantum interference device (SQUID) (See S. Tanaka, et al, “A DNA Detection System Based Upon a High Tc SQUID and Ultra-Small Magnetic Particles”, IEEE Transaction on Applied Superconductivity, Vol 15, No. 2, June 2005), giant magnetoresistance (GMR) arrays (See G. Li, S. Wang, and S. Sun, “Model and Experiment of Detecting Multiple Magnetic Nanoparticles as Biomolecular Labels by Spin Valve Sensors”, IEEE Trans.on Magnetics, vol. 40, No. 4, pp. 3000-3002, July 2004) and atomic force microscopy (See G. A. Gibson and S. Schultz, “Magnetic force microscope study of the micromagnetics of submicrometer magnetic particles”, Journal of Applied Physics, vol. 73, issue 9, pp. 4516-4521, January 1993) are used for their high sensitivity. However, these sensing methods either cannot be integrated or require high-cost post-processing steps, which limits their popularities. Hall sensors (See T. Aytur, P. R. Beatty, and B. Boser, “An Immunoassay Platform Based on CMOS Hall Sensors”, Solid-State Sensor, Actuator and Microsystems Workshop, June 2002 and P. A. Besse, G Boero, M. Demierre, V. Pott, and R. Popovic, “Detection of a single magnetic microbead using a miniaturized silicon Hall sensor”, Applied Physics Letters, vol. 80, No. 22, pp. 4199-4201, June 2002) are available in standard CMOS process, but they require external biasing field that is demanding as regards power, reducing the applicability of Hall sensors in portable systems and limiting the compatibility of Hall sensor systems with micro-fluidic systems. In addition, Hall sensors need to be of comparative dimensions (sensor size and passivation layer thickness) with respect to the sensed magnetic beads for optimum sensitivity. This limits the use of Hall sensors to very small sensing area without compatibility with different sizes of magnetic particles.

Issues for conventional cellular analysis methods is that their sensing approaches only deal with the ensemble responses of all the cells present, rather than responses from individual cells or even small groups of cells. This issue significantly limits the information content that those approaches can provide and the applications they can support, because they mask local information of the cells to be tested.

There is a need for systems and methods to investigate cells using a portable laboratory-on-a-chip configuration.

### SUMMARY OF THE INVENTION

According to one aspect, the invention features an electromagnetic cellular tomograph. The electromagnetic cellular tomograph comprises an electromagnetic structure configured to apply a probe signal to a sample of interest and to receive a response signal from the sample of interest, the electromagnetic structure having at least one input terminal connected to an electromagnetic signal application structure and having at least one output terminal connected to an electromagnetic signal sensing structure; a source circuit configured to provide an electromagnetic probe signal, the source circuit having at least one output terminal in electrical communication with the at least one input terminal of the electromagnetic structure configured to apply the probe signal to the at least one input terminal of the electromagnetic structure, and having at least one control input terminal configured to receive a control signal; a sensing circuit configured to sense the response signal from the sample of interest, the sensing circuit having at least one input terminal to receive the response signal from the at least one output terminal of the electromagnetic structure, the sensing circuit having at least one output terminal at which the sensed response signal is provided; a control circuit having at least one terminal in electrical communication with the at least one control input terminal of the source circuit, having at least one terminal in electrical communication with the at least one output terminal of the sensing circuit, and having at least one pair of input and output terminals for communication with an external electrical apparatus, the control circuit configured to control the source circuit, and the control circuit configured to communicate the sensed signals from the sensing circuit to the external electrical apparatus, and to accept control signals the external electrical apparatus; and a sample holder adjacent the electromagnetic structure, the sample holder configured to hold a sample of interest.

In one embodiment, the electromagnetic structure is fabricated on a single chip.

In another embodiment, the electromagnetic structure is configured as an N×M array, N and M being integers greater than or equal to 1.

In yet another embodiment, the electromagnetic structure is configured to apply a probe signal comprising an electrical current to the sample of interest.

In still another embodiment, the electromagnetic structure is configured to apply a probe signal comprising an electrical voltage to the sample of interest.

In a further embodiment, the electromagnetic structure is configured to apply a probe signal comprising an electrical field to the sample of interest.

In another embodiment, the electromagnetic structure is configured to apply a probe signal comprising a magnetic field to the sample of interest.

In yet another embodiment, the electromagnetic structure is configured to apply a probe signal comprising a mechanical force arising from an electrostatic field or a magnetostatic field to the sample of interest.

According to another aspect, the invention relates to an electromagnetic cellular tomography method. The method comprises the steps of providing an electromagnetic cellular tomograph, comprising: an electromagnetic structure configured to apply a probe signal to a sample of interest and to receive a response signal from the sample of interest, the electromagnetic structure having at least one input terminal connected to an electromagnetic signal application structure and having at least one output terminal connected to an electromagnetic signal sensing structure; a source circuit configured to provide an electromagnetic probe signal, the source circuit having at least one output terminal in electrical communication with the at least one input terminal of the electromagnetic structure configured to apply the probe signal to the at least one input terminal of the electromagnetic structure, and having at least one control input terminal configured to receive a control signal; a sensing circuit configured to sense the response signal from the sample of interest, the sensing circuit having at least one input terminal to receive the response signal from the at least one output terminal of the electromagnetic structure, the sensing circuit having at least one output terminal at which the sensed response signal is provided; a control circuit having at least one terminal in electrical communication with the at least one control input terminal of the source circuit, having at least one terminal in electrical communication with the at least one output terminal of the sensing circuit, and having at least one pair of input and output terminals for communication with an external electrical apparatus, the control circuit configured to control the source circuit, and the control circuit configured to communicate the sensed signals from the sensing circuit to the external electrical apparatus, and to accept control signals the external electrical apparatus; and a sample holder adjacent the electromagnetic structure, the sample holder configured to hold a sample of interest. The method includes the steps of providing a sample of interest in the sample holder; applying a probe signal to the sample of interest; sensing a response signal from the sample of interest; analyzing the response signal to obtain a result; and performing at least one of recording the result, transmitting the result to a data handling system, or to displaying the result to a user.

In one embodiment, the probe signal comprises an electrical current.

In another embodiment, the probe signal comprises an electrical voltage.

In yet another embodiment, the probe signal comprises an electrical field.

In still yet another embodiment, the probe signal comprises a magnetic field.

In a further embodiment, the probe signal comprises a mechanical force arising from an electrostatic field or a magnetostatic field.

The foregoing and other objects, aspects, features, and advantages of the invention will become more apparent from the following description and from the claims.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1 is a schematic diagram illustrating a sensor approach including its major building blocks as components.

FIG. 2 is a schematic diagram illustrating a stimulator approach with its major building blocks as components.

FIG. 3 is a schematic diagram illustrating a sensor based on electrical impedance measurements.

FIG. 4 is a schematic diagram illustrating a bridge for electrical impedance measurement.

FIG. 5 is a schematic diagram illustrating a stimulation system using electrical current.

FIG. 6 is a schematic diagram illustrating a stimulation system using electrical voltage.

FIG. 7 is a schematic diagram illustrating a stimulation system using magnetostatic force stimulation.

FIG. 8 is a schematic diagram illustrating a differential sensor approach.

FIG. 9 is a schematic diagram illustrating a 2D M×N dimensional sensor/stimulator array.

### DETAILED DESCRIPTION

This invention describes a novel electromagnetic cellular tomography approach, which can be used to, but is not limited to, the study and analysis of cell-to-cell interactions and tissue formations. By using integrated technology, a high pixel-density and high sensitivity scalable probe array can be formed to sense and/or stimulate the cells/tissues under test. The change of the cellular electromagnetic properties can be sensed to infer the corresponding biological properties changes. In addition, programmable electromagnetic/mechanical stimuli can also be applied to the cells to selectively enhance or suppress certain cellular developments. While the traditional methods are limited to process the ensemble response from all the cells, this approach provides the detailed property information of and controllable stimulation access to individual cells or even certain parts of the cell under test, which can serve as a general platform for lab-on-a-chip (LOC) cellular studies. In any of embodiments that are described, a general purpose programmable computer programmed with suitable software (e.g., instructions in machine readable form recorded on a machine readable medium) can be used to control the operation of the tomograph, to receive data, to manipulate and/or analyze the data, and to record results, transmit the results to another data handling system, or to display results to a user.

Tomography is commonly used to describe a technique in x-ray methods, in which a two-dimensional image of a slice or section is taken through a three-dimensional object. Here, we present a similar method that provides two-dimensional images or data taken on individual cells or groups of cells (“tissues”). The term “tomogram” is used to refer to an image or picture; the term “tomograph” is used to refer to an apparatus; and the term “tomography” is used to refer to a process.

It is expected that micro/nano magnetic beads be engineered to be fully biocompatible and can be made available with most commonly used bio-probe coating materials, which is expected to make them capable to serve as a new sensing platform.

In this disclosure, we describe an electromagnetic cellular tomography approach which relies on high pixel-density and high precision electromagnetic sensor arrays to provide detailed property information of and controllable stimulation access to every individual cell under test. This localized sensing/excitation capability of our approach opens the door for a plethora of comprehensive cellular studies. These include cell-to-cell communications, cellular behaviors under electrical/magnetic/chemical/mechanical stimuli and tissue formation, which can be directly utilized in applications as testing the efficacy of a new drug, the efficacy of a drug for a given cell type or individual, and artificial tissue cultivation. More importantly, our approach is completely compatible with commercial integrated technology, such as CMOS, and does not require any optical instruments. Therefore, the implementation of our approach can achieve a scalable Lab-on-a-Chip (LOC) system for cellular studies, and diagnostic testing, with low cost and small form-factor.

This disclosure is organized in two main parts. Our sensing/stimulating approach is presented in detail in the first section. Then, as implementation examples, several embodiments, including sensor cell topologies and sensor system architectures are described.

### General Description of the Cellular Sensing/Stimulating Approach

In this section, we discuss a new sensing/stimulating approach, electromagnetic cellular tomography, which is useful for advanced cellular studies.

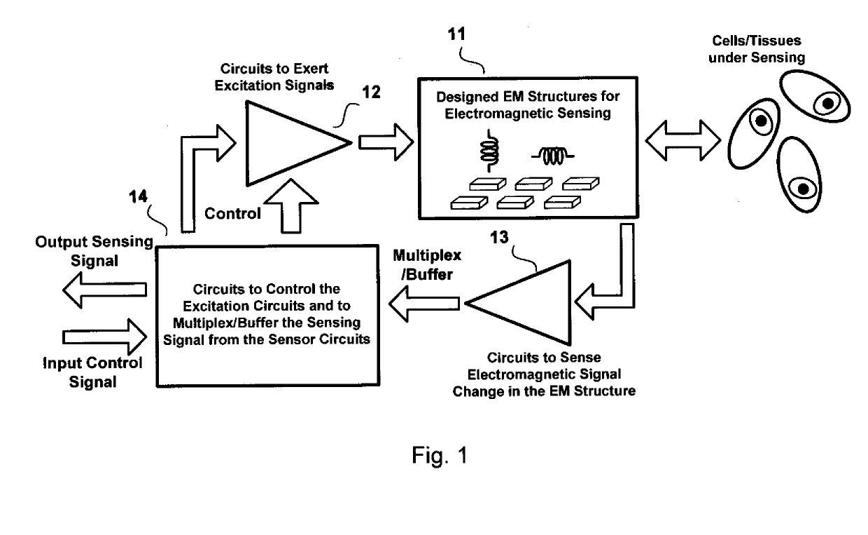

FIG. 1 illustrates a generalized sensing device and the approach we use in terms of the following components configured in a loop. Block 11 is an optimized electromagnetic (EM) structure for electrical and/or magnetic sensing. Block 11 can include one or a plurality of structures. The cells or tissues to be examined interact with Block 11. Block 12 represents one or more excitation circuits that apply excitation signals (“probe signals”) to the optimized EM structures of Block 11. Block 13 represents one or more sensing circuits that can sense changes in signals (“response signals”) from the optimized EM structures of Block 11 as the properties of the optimized EM structures change. Block 14 represents one or more circuits configured to control the one or more excitation circuits of Block 12, to communicate the sensed response signals from the sensor circuits of Block 13 to an off-chip environment (for example an external electrical apparatus such as a computer), and to accept control signals from the off-chip environment. Block 14 can include such elements as multiplexers, buffers, and conversion circuits to provide suitable bidirectional interfaces between the electromagnetic cellular tomography sensing device and external circuits.

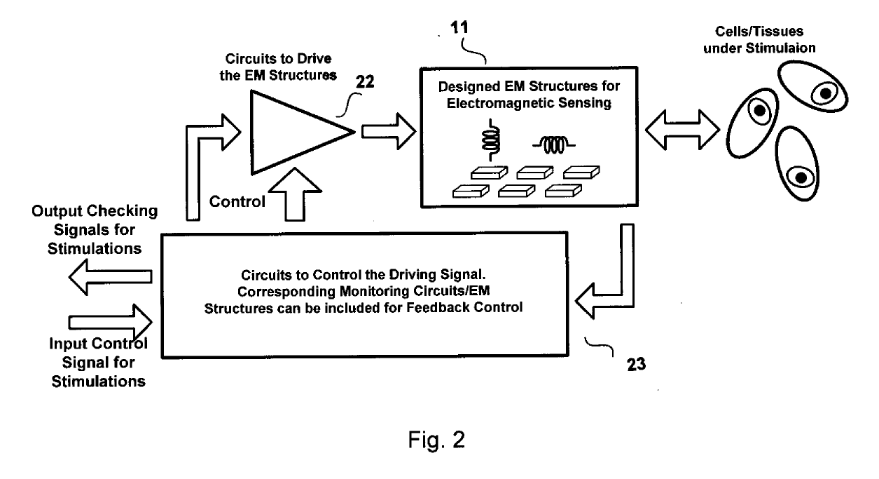

FIG. 2 is a schematic diagram illustrating a generalized stimulator approach with its major building blocks as components configured in a loop. Block 11 is an optimized electromagnetic (EM) structure for electrical and/or magnetic sensing. Block 11 can include one or a plurality of structures. The cells or tissues to be examined interact with Block 11. Block 22 represents one or more excitation circuits that apply excitation signals to the optimized EM structures of Block 11. Block 23 represents one or more circuits configured to control the one or more excitation circuits of Block 22, to communicate the sensing signals from the sensor circuits of Block 11 to an off-chip environment, and to accept control signals from the off-chip environment. Block 23 can include such elements as multiplexers, buffers, and conversion circuits to provide suitable bidirectional interfaces between the electromagnetic cellular tomography sensing device and external circuits. Block 23 in some embodiments preferably includes circuits for monitoring the EM structures/circuits. The circuits can also be implemented for feedback purposes.

The cells or tissues to be examined can be any of natural cells, wild-type cells/tissues, or modified cells/tissues. The modified cells or tissues, for example, can possess specific ion channels/pumps for electrical sensing/stimulating or antibodies labeled with magnetic particle labeling for magnetic sensing/stimulating.

The working mechanism for both the sensor approach and the stimulator approach will be described with an emphasis on how the components function and interact with each other.

In the sensing system, the sensor units are designed and laid-out with high spatial density to minimize pixel size. The sensing circuits generate required signals to interact with the designed EM structures, which detect the electrical/magnetic properties of the cells or tissues that are being tested. These properties include, but are not limited to, RC impedance sensing and/or effective inductance sensing. The multiplexing/buffering circuits scan the individual sensors in the sensor array having one or more sensors to send the detected signals off-chip. Cells experience various property changes during responses to cofactors and tissue formation. By way of example, dislocation of the ion channels/pumps and labeled magnetic particles can be sensed by corresponding change of electrical and magnetic signals for that particular cell. Changes in the electrical/magnetic signals of an entire region may occur when cells aggregate and form a closely packed tissue. The changes in the electrical/magnetic signals of the entire region can be detected by the corresponding sensor pixels adjacent such aggregating cells.

In our stimulating structure, the driving circuit generates electrical signals that are appropriate to apply stimuli to cells and tissues through the high-density EM structures. In various embodiments, the stimuli preferably can be electrical currents, electrical potentials or electrical fields, magnetic fields, or mechanical forces that are applied through electromagnetic interactions, such as electrostatic and magnetostatic effects. In general, a monitoring structure is preferably implemented to provide a feedback loop, so that the stimuli applied to the cells or tissues can be controlled with regard to the amplitude and/or the phase of the stimuli applied to the cells or tissues.

The sensor array and the stimulator array each can be provided as high density arrays. Our approach provides access to individual cells as well as providing a larger sensing/stimulating area for characterization of groups of cells. In this approach, the apparatus provides an electromagnetic cellular tomograph which allows a user to study cells and tissues with both high spatial density and highly accurate individual cellular examinations.

Practical implementation examples of the sensor and stimulator component parts are presented in the next section. In a practical design, the component parts may not be implemented as separate blocks. For example, in some embodiments, a specific block in a sensor may include both the functions of applying excitations signals and sensing the cellular response simultaneously.

### Illustrative Implementations of the Sensor and Stimulator

Based on our approach, there are various ways to implement the sensor, the stimulator, and the entire tomography system. We present several embodiments below. The practical implementation of our approach can comprise, but is not limited to, the illustrative examples shown. While some embodiments are illustrated, other cellular study system embodiments that use our approach are contemplated.

### EXAMPLE 1

### Impedance Measurement for Electrical Sensing

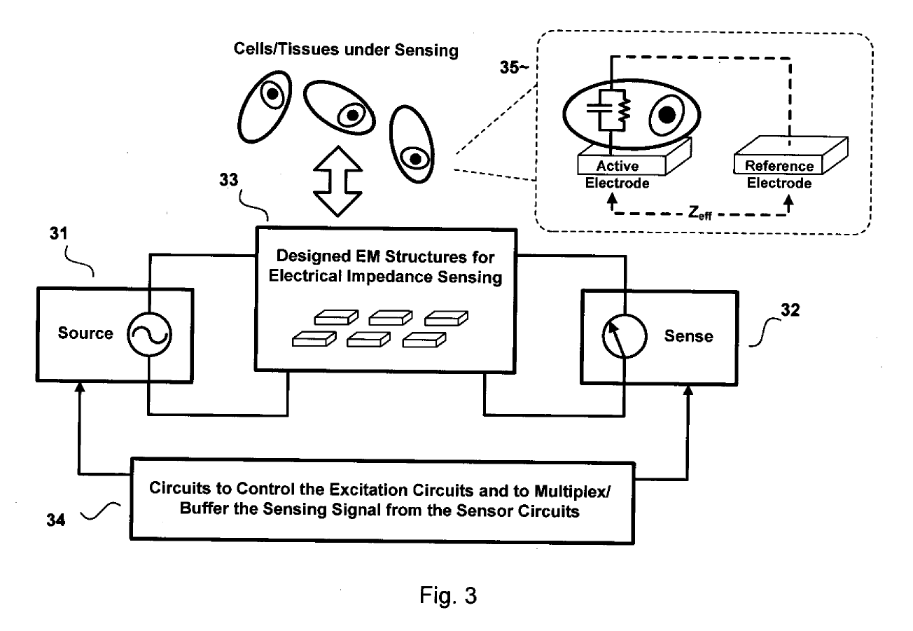

Circuits and EM structures can be designed to measure the local impedance of the cell directly. A basic impedance measurement system is shown in FIG. 3.

The signal source element 31 and the signal sense element 32 respectively provide and sense signals that can be any of voltage, current or power. The source signals and the sense signals can be narrowband, tunable narrowband or broadband in nature. Block 33 is a sensing block which interacts with the cells. It can comprise one or several electrodes or any EM structures which provide active terminal(s) and reference terminal(s). The impedance of Block 33 will be highly dependent on the behavior of one or more cells adjacent the block 33, and can vary both in amplitude and in phase. Block 34 controls the source signal provided by Block 31 and multiplexes/buffers the measured signal that is provided as output by sensor block 32.

As illustrated in the diagram 35 at the upper right of FIG. 3, in some embodiments preferably a reference electrode and an active electrode are used together. The reference electrode is separate from the cell or tissue to be examined, and the active electrode is adjacent the cell or tissue to be examined. Measurement of an output signal involves a differential measurement across the two electrodes.

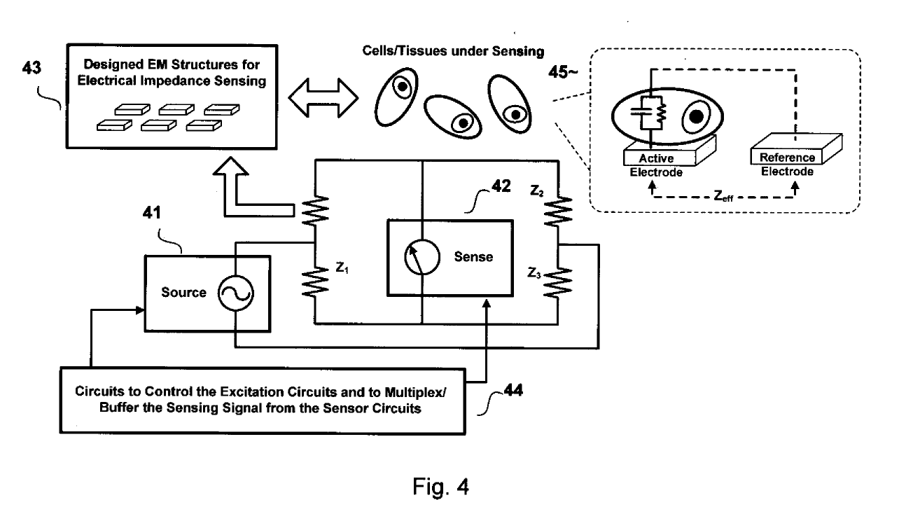

FIG. 4 is a schematic diagram illustrating a bridge for electrical impedance measurement. One implementation for this is shown in FIG. 4. Block 43 in FIG. 4 represents the EM structure which interfaces with the cell samples with its active/reference electrodes. In block 42, impedances Z₁, Z₂ and Z₃ are provided for matching purposes. Block 41 represents the excitation source while Block 42 reads out the voltage or current signal change, which indicates the impedance change in Block 43. Block 44 controls the source signal and multiplexes/buffers the measured output signal. Block 45 is analogous to Block 35 in FIG. 3.

### EXAMPLE 2

### Magnetic Sensing

Magnetic sensing can be performed directly on magnetic cells like magnetic bacteria or on cells with magnetic particle labels. Since the detailed sensing method and implementation has been described extensively in the prior art literature, we will briefly list them for completeness.

### Effective Inductance Change Based Magnetic Sensing

This sensing method utilizes on-chip effective inductance change to probe the existence of nearby magnetic materials. It provides high sensor accuracy without using external biasing magnetic field and/or expensive post processes. The detailed implementation method has been described in U.S. patent application Ser. No. 12/399,603 filed Mar. 6, 2009, U.S. patent application Ser. No. 12/559,517 filed Sep. 15, 2009, U.S. patent application Ser. No. 12/710,334 filed Feb. 22, 2010, and H. Wang and A. Hajimiri, “Design of Inductors with Uniform Magnetic Field Strength in the Near-Field,” U.S. Provisional patent application Attorney Docket No. CIT-5505-P filed Dec. 23, 2009.

### Other Magnetic Sensing Implementations

There are other methods to implement magnetic sensors including Hall sensors, spin valve sensors, and Superconducting Quantum Interference Device (SQUID) systems. These sensors can potentially achieve very high pixel densities using an external biasing magnetic field.

### EXAMPLE 3

### Programmable Electrical Current Sources for Electrical Stimulus

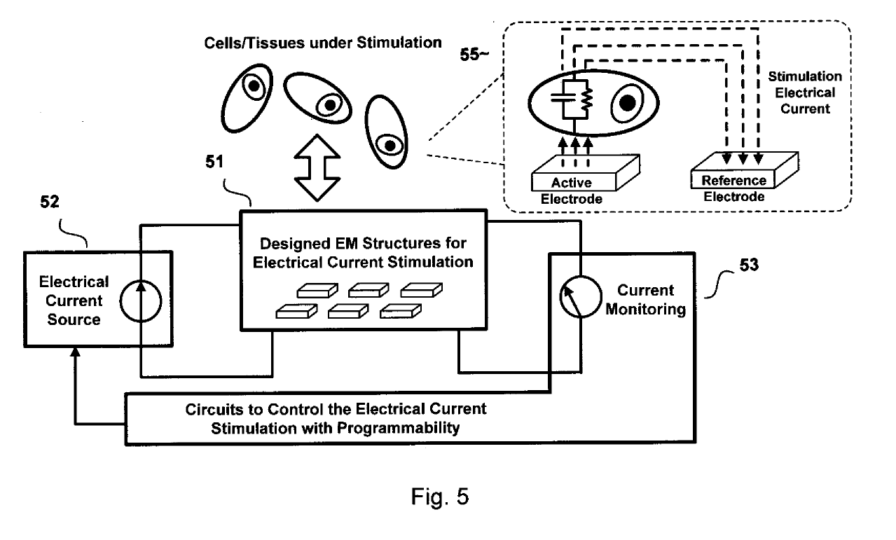

Electrical currents can be used as stimuli for the cells under study. FIG. 5 is a schematic diagram illustrating a stimulation system using electrical current.

In FIG. 5, Block 52 represents circuits that are current sources. Preferably, high output impedance is advantageous for Block 52 to minimize the loading effect from the biological media. The electrical current from Block 52 passes through the cell and its surrounding environment and returns back to the reference electrode to close the circulating loop, as is illustrated in Block 55 in the upper right of FIG. 5. Block 51 represents a designed EM structure, comprising electrodes, which serve as the interface between on-chip electronics and the biological media for conducting the currents. Block 53 monitors the output currents from the electrodes. Block 53 preferably includes circuitry to provide a feedback loop, so that the stimuli applied to the cells or tissues can be controlled with regard to their amplitude and/or their phase. In some embodiments, the feedback circuitry is programmable, and can be controlled using a general purpose programmable computer programmed with suitable software (e.g., instructions in machine readable form recorded on a machine readable medium). In some embodiments, the programmable feedback circuitry can be implemented using conventional PID control technology, which can be implemented in hardware, or in software. One way to implement Block 53 is through sensing voltages across a sensing resistor connected between a reference electrode and a chip ground. By knowing the resistor value R a priori, the voltage V across the resistor directly provides a measure of the current flowing through it (e.g., 1=V/R), which is also the current stimulus from Block 52. Since Block 52 is preferably designed with high output impedance, the sensing resistor (normally designed with much smaller impedance value compared with the impedance of the media if in series, and normally designed with a much larger impedance if in parallel will present a negligible loading on Block 52 and will not affect the actual current stimulus output. Block 55 is analogous to Block 35 in FIG. 3.

### EXAMPLE 4

### Programmable Electrical Voltage Sources for Electrical Stimulus

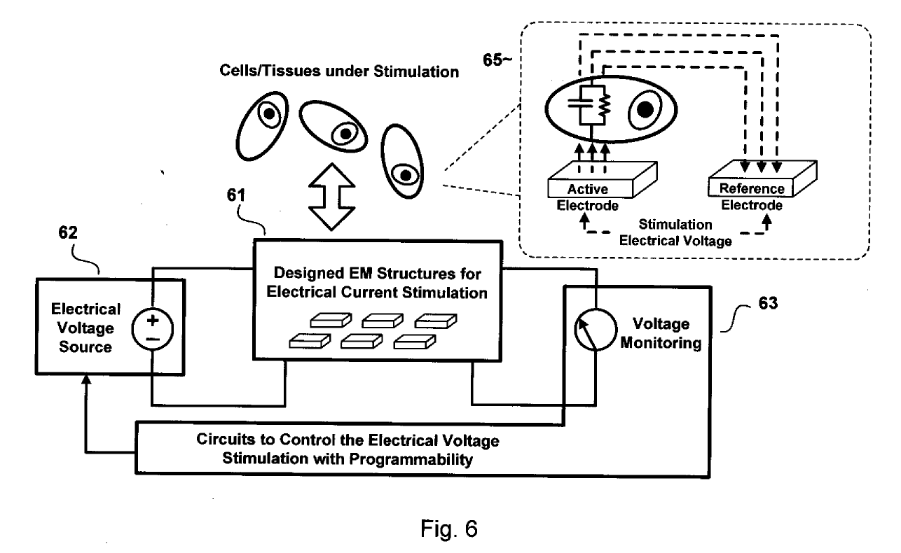

Electrical voltage also can be used as a stimulus. FIG. 6 is a schematic diagram illustrating a stimulation system using electrical voltage.

In FIG. 6, Block 62 represents circuits functioning as voltage sources. Preferably, a low output impedance is advantageous for Block 62 to minimize the loading effect and to allow Block 62 to provide a good voltage driver. The electrical current from Block 62 passes through the cell and its surrounding environment and return back to the reference electrode to close the circulating loop. Block 61 indicates a designed EM structure, comprising electrodes, which serve as the interface between on-chip electronics and the biological media for conducting the currents. The circulating current builds up the desired voltage between the active electrode and the reference electrode. Block 63 monitors the excitation voltage as a feedback signal to control at least a selected one of the amplitude and the phase of the current stimulus. One way to implement Block 63 is through sensing potential difference between the active electrode and the reference electrode directly. Since Block 62 provides a small output impedance, this voltage sensing circuitry (normally designed with much smaller impedance value compared with the impedance of the media if in series, and normally designed with a much larger impedance if in parallel), presents negligible loading toward the Block 62 and will not affect the actual voltage stimulus output. Block 65 is analogous to Block 35 in FIG. 3.

### EXAMPLE 5

### Magnetostatic-Based Mechanical Stimulus

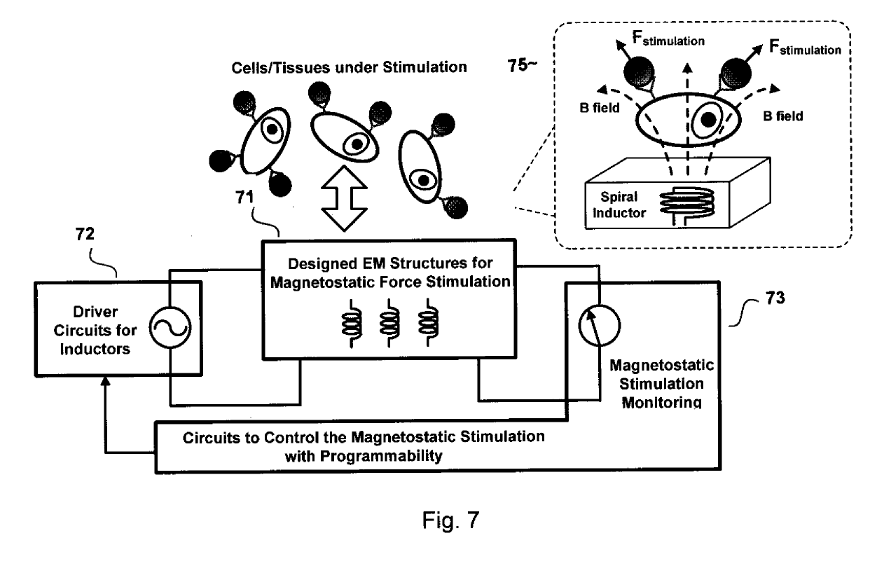

FIG. 7 is a schematic diagram illustrating a stimulation system using magnetostatic force stimulation. Magnetic field exerts mechanical forces on materials with magnetic properties. This force can be utilized as mechanical stimulus for cellular studies.

Block 71 indicates a designed EM structure, for example comprising an on-chip spiral inductor, which generates the target magnetic field strength when conducting certain DC/AC current generated by circuit Block 72. In some embodiments, Block 73 preferably contains both open-loop settings and feedback controls to control the applied B field with the target amplitude/phase value. Block 75 illustrates the physics of magnetic fields as they are applied to the cells or tissues that are being examined.

### Structures for Delivering Samples to the Tomograph

Depending on different applications, cell/tissue samples for cellular study vary significantly both in quantity and in type. Therefore, there are many possible implementations of the sample delivering system. These can include several embodiments, which are all compatible with the aforementioned sensor/stimulator designs. The samples can be delivered via a sub-pt (micro-Liter) volume pipette controlled by a stepper motor having a fine step. This is suitable for a case when a large number of samples are needed to be delivered. Alternatively, a microfluidic channel can be designed to deliver the sample which also forms an enclosed environment for the sample adjacent the EM structure. A bonding technique to attach the PDMS microfluidic device to the integrated chips is described in U.S. patent application Ser. No. 12/713,128. In other embodiments, optical tweezers can be used to deliver individual cells or small piece of tissue. This is suitable for applications when a very small amount of a sample needs to be delivered with high spatial accuracy.

### Implementation of the Sensor/Stimulator System

We now present several approaches to implement the sensor/stimulator based of our Electromagnetic Cellular Tomograph described hereinabove.

### Differential Sensing Approach

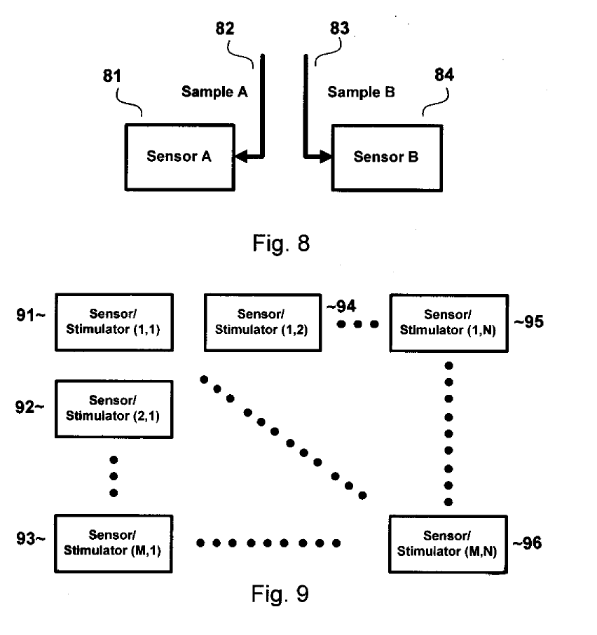

FIG. 8 is a schematic diagram illustrating a differential sensor approach.

Block 81 and Block 84 represent two sensors implemented in any of the formats previously described. Preferably, they should be of the same type. Preferably, they should be put close to each other in physical layout to improve the matching between the two sensors in order to provide similar sensor response, for example with regard to external stimuli, such as temperature or pressure. Arrow 82 and arrow 83 indicate the delivery of different samples, i.e., sample A and sample B, respectively to each of sensors 81 and 84. In one embodiment, sensor A (block 81) is used as a main sensor and sensor B (block 84) is used as a reference sensor. Differential sensing can proceed as follows.

Sample A is delivered to Sensor A, and reference sample B is delivered to reference sensor B. The response of sensor A and sensor B can be recorded separately. The signal difference in the two sensor responses can be calculated to obtain a differential sensing result.

By having differential sensing, any common-mode offset of the sensor response will be eliminated, as long as good matching is preserved between the two sensors. Those non-ideal offsets include temperature drifting, power supply noise and common (non-specific) cellular electromagnetic signals. There can be multiple main sensors and multiple reference sensors. In some embodiments, the role of the main sensor and the reference sensor can be interchanged. In other words, we can use sensor A as the main sensor and sensor B as the reference and do the sensing procedures mentioned above to get a first differential signal result (which will be termed Result 1). We can use sensor B as the main sensor and sensor A as the reference to get a second differential signal result (which will be termed Result 2). The two results can be averaged to further eliminate the systematic offsets in sensor response. The two results (Result 1 and Result 2) can be obtained in any order. In addition, the reference sample can also be only buffer solutions (or even an empty sensor) with no cells/tissues present.

### Sensor/Stimulator Array

The Electrical Cellular Tomograph can be easily extended to an array structure, shown in FIG. 9. FIG. 9 is a schematic diagram illustrating a 2D M×N dimensional sensor/stimulator array. N and M are integers greater than or equal to 1. Every block 91, 92, 93, 94, 95, 96 represents a sensor/stimulator implemented as described hereinabove. The sensor/stimulator unit cells can be of the same or different types. The 2D array that is depicted can be reduced to a 1D array or extended to a 3D array if fabrication and packaging technologies permit. The sensor/stimulator array can be fabricated on a single chip, on multiple chips, or on a complete discrete basis.

An advantage of a sensor/stimulator array is that it improves the system throughput by a significant factor. Applications of the sensor/stimulator array include, but not limited to, the following examples.

The incoming samples can be sensed or stimulated differently by using different type sensor/stimulator cells in the array. In this situation, the M×N sensor/stimulator array will process the same type of samples by M×N methods simultaneously. This high throughput allows comprehensive study and comparison of the same type of samples via different sensing/stimulating techniques.

Multiple types of samples can be processed by the sensor/stimulator array. In this case, with an M×N array, M×N types of sample can be sensed simultaneously. This allows study and comparison of different sample types.

Functionalities of different biochemical signals, e.g., cofactors, can also be tested by the array. In this case, a microfluidic system should have the capability for individually addressing different sensor/stimulator cells or groups. Therefore, with an M×N array, maximally M×N types of biochemicals can be tested to investigate their effects on the cell/tissue samples.

Combinations of the approaches described may be used to achieve overall versatile functionalities. The aforementioned variations in sensor/stimulator implementation, e.g. differential sensing approach and array format, are not exclusive to each other. Based on specific application, they can be combined to form optimized Electromagnetic Cellular Tomography system to achieve a fully integrated and battery powered lab-on-a-chip (LOC) cellular study system with extremely low cost and ultra portability.

It is believed that the electromagnetic cellular tomograph can be fabricated by standard microelectronics foundry processes including , but not limited to, CMOS or BiCMOS as Si-based processes or GaAs or GaN as III-V compound processes, and/or various micromachining processes, such as MEMS (Microelectromechanical systems) or (Nanoelectromechanical systems) NEMS processes.

### Clinical Application for the Early Detection and Diagnosis of Cancer and Cancer Metastasis

With an increased average lifespan, tumor formation and cancer metastasis have become one of the life threatening issues for mankind. Cancer has been the second leading cause of death in United States (See American Cancer Society, Inc. Cancer Facts and Figures 2005. Atlanta: American Cancer Society, Inc., 2005). It is believed that the formation of cancer is caused by changes in genes that normally control the growth and death of cells. Many of such genetic changes result from tobacco use, diet, exposure to ultraviolet (UV) radiation from the sun, or exposure to carcinogens (cancer-causing substances) in the workplace or in the environment. This occurs in a sporadic, unpredictable way, making the detection and diagnosis of cancer in its early stages intractable.

In normal human bodies, it is believed that the proliferation, death, and migration of cells is regulated by their interaction with other cells, extracellular matrix, or soluble factors such as cytokines and growth factors, through their surface receptors (See Guo, W. & Giancotti, F. G. Integrin signaling during tumor progression. Nat Rev Mol Cell Biol 5, 816-826. (2004)). Thus, one potential indicator for tumor formation or cancer metastasis is the abnormal expression of surface molecules. A sensitive and efficient screening tool to identify one single abnormal cell in a large cell population would help to detect tumors in their early stages, given that the formation of tumors is sporadic. Current diagnosis tools rely on the single-cell sorting/detection technique. For example, to detect the cancerous cell, individual cells from a large cell population will be enzymatically isolated, labeled with fluorescent or magnetic probes (See H. Lee, E. Sun, D. Ham, and R. Weissleder, “Chip-NMR biosensor for detection and molecular analysis of cells,” Nature Medicine, vol. 14, no. 8, pp. 869-874, August 2008), and then screened using optical or magnetic instruments. This is an exhausting, time-consuming search, with no a priori assurance that it will provide any useful information in any specific instance. The expense can be very high; yet the efficiency might be low or unsatisfactory. Moreover, the single-cell isolation technique destroys the integrity of tissue, which can contain useful clinical information. By comparison, our method is efficient, low-cost, and yet sufficiently sensitive. Furthermore, our method allows for the preservation of the entire tissue because single-cell isolation is not required. These advantages make our device an ideal tool for the detection and diagnosis of cancer and cancer metastasis in the early stage.

### Definitions

Recording the results from an imaging operation or image acquisition, such as for example, recording results at a particular wavelength, is understood to mean and is defined herein as writing output data to a storage element, to a machine-readable storage medium, or to a storage device. Machine-readable storage media that can be used in the invention include electronic, magnetic and/or optical storage media, such as magnetic floppy disks and hard disks; a DVD drive, a CD drive that in some embodiments can employ DVD disks, any of CD-ROM disks (i.e., read-only optical storage disks), CD-R disks (i.e., write-once, read-many optical storage disks), and CD-RW disks (i.e., rewriteable optical storage disks); and electronic storage media, such as RAM, ROM, EPROM, Compact Flash cards, PCMCIA cards, or alternatively SD or SDIO memory; and the electronic components (e.g., floppy disk drive, DVD drive, CD/CD-R/CD-RW drive, or Compact Flash/PCMCIA/SD adapter) that accommodate and read from and/or write to the storage media. As is known to those of skill in the machine-readable storage media arts, new media and formats for data storage are continually being devised, and any convenient, commercially available storage medium and corresponding read/write device that may become available in the future is likely to be appropriate for use, especially if it provides any of a greater storage capacity, a higher access speed, a smaller size, and a lower cost per bit of stored information. Well known older machine-readable media are also available for use under certain conditions, such as punched paper tape or cards, magnetic recording on tape or wire, optical or magnetic reading of printed characters (e.g., OCR and magnetically encoded symbols) and machine-readable symbols such as one and two dimensional bar codes. Recording image data for later use (e.g., writing an image to memory or to digital memory) can be performed to enable the use of the recorded information as output, as data for display to a user, or as data to be made available for later use. Such digital memory elements or chips can be standalone memory devices, or can be incorporated within a device of interest. “Writing output data” or “writing an image to memory” is defined herein as including writing transformed data to registers within a microcomputer.

“Microcomputer” is defined herein as synonymous with microprocessor, microcontroller, and digital signal processor (“DSP”). It is understood that memory used by the microcomputer, including for example an imaging or image processing algorithm coded as “firmware” can reside in memory physically inside of a microcomputer chip or in memory external to the microcomputer or in a combination of internal and external memory. Similarly, analog signals can be digitized by a standalone analog to digital converter (“ADC”) or one or more ADCs or multiplexed ADC channels can reside within a microcomputer package. It is also understood that field programmable array (“FPGA”) chips or application specific integrated circuits (“ASIC”) chips can perform microcomputer functions, either in hardware logic, software emulation of a microcomputer, or by a combination of the two. Apparatus having any of the inventive features described herein can operate entirely on one microcomputer or can include more than one microcomputer.

General purpose programmable computers useful for controlling instrumentation, recording signals and analyzing signals or data according to the present description can be any of a personal computer (PC), a microprocessor based computer, a portable computer, or other type of processing device. The general purpose programmable computer typically comprises a central processing unit, a storage or memory unit that can record and read information and programs using machine-readable storage media, a communication terminal such as a wired communication device or a wireless communication device, an output device such as a display terminal, and an input device such as a keyboard. The display terminal can be a touch screen display, in which case it can function as both a display device and an input device. Different and/or additional input devices can be present such as a pointing device, such as a mouse or a joystick, and different or additional output devices can be present such as an enunciator, for example a speaker, a second display, or a printer. The computer can run any one of a variety of operating systems, such as for example, any one of several versions of Windows, or of MacOS, or of UNIX, or of Linux. Computational results obtained in the operation of the general purpose computer can be stored for later use, and/or can be displayed to a user. At the very least, each microprocessor-based general purpose computer has registers that store the results of each computational step within the microprocessor, which results are then commonly stored in cache memory for later use.

Many functions of electrical and electronic apparatus can be implemented in hardware (for example, hard-wired logic), in software (for example, logic encoded in a program operating on a general purpose processor), and in firmware (for example, logic encoded in a non-volatile memory that is invoked for operation on a processor as required). The present invention contemplates the substitution of one implementation of hardware, firmware and software for another implementation of the equivalent functionality using a different one of hardware, firmware and software. To the extent that an implementation can be represented mathematically by a transfer function, that is, a specified response is generated at an output terminal for a specific excitation applied to an input terminal of a “black box” exhibiting the transfer function, any implementation of the transfer function, including any combination of hardware, firmware and software implementations of portions or segments of the transfer function, is contemplated herein.

### Theoretical Discussion

Although the theoretical description given herein is thought to be correct, the operation of the devices described and claimed herein does not depend upon the accuracy or validity of the theoretical description. That is, later theoretical developments that may explain the observed results on a basis different from the theory presented herein will not detract from the inventions described herein, so long as at least some of the implementation is performed in hardware.

Any patent, patent application, or publication identified in the specification is hereby incorporated by reference herein in its entirety. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

While the present invention has been particularly shown and described with reference to the preferred mode as illustrated in the drawing, it will be understood by one skilled in the art that various changes in detail may be affected therein without departing from the spirit and scope of the invention as defined by the claims.

What is claimed is:

1. An electromagnetic cellular tomograph, comprising:
an electromagnetic structure configured to apply a probe signal to a sample of interest and to receive a response signal from said sample of interest, said electromagnetic structure having at least one input terminal connected to an electromagnetic signal application structure and having at least one output terminal connected to an electromagnetic signal sensing structure;
a source circuit configured to provide an electromagnetic probe signal, said source circuit having at least one output terminal in electrical communication with said at least one input terminal of said electromagnetic structure configured to apply said probe signal to said at least one input terminal of said electromagnetic structure, and having at least one control input terminal configured to receive a control signal;
a sensing circuit configured to sense said response signal from said sample of interest, said sensing circuit having at least one input terminal to receive said response signal from said at least one output terminal of said electromagnetic structure, said sensing circuit having at least one output terminal at which said sensed response signal is provided;
a control circuit having at least one terminal in electrical communication with said at least one control input terminal of said source circuit, having at least one terminal in electrical communication with said at least one output terminal of said sensing circuit, and having at least one pair of input and output terminals for communication with an external electrical apparatus, said control circuit configured to control said source circuit, and said control circuit configured to communicate the sensed signals from the sensing circuit to said external electrical apparatus, and to accept control signals said external electrical apparatus; and
a sample holder adjacent said electromagnetic structure, said sample holder configured to hold a sample of interest.

2. The electromagnetic cellular tomography of claim 1, wherein said electromagnetic structure is fabricated on a single chip.

3. The electromagnetic cellular tomography of claim 1, wherein said electromagnetic structure is configured as an N×M array, N and M being integers greater than or equal to 1.

4. The electromagnetic cellular tomography of claim 1, wherein said electromagnetic structure is configured to apply a probe signal comprising an electrical current to said sample of interest.

5. The electromagnetic cellular tomography of claim 1, wherein said electromagnetic structure is configured to apply a probe signal comprising an electrical voltage to said sample of interest.

6. The electromagnetic cellular tomography of claim 1, wherein said electromagnetic structure is configured to apply a probe signal comprising an electrical field to said sample of interest.

7. The electromagnetic cellular tomography of claim 1, wherein said electromagnetic structure is configured to apply a probe signal comprising a magnetic field to said sample of interest.

8. The electromagnetic cellular tomography of claim 1, wherein said electromagnetic structure is configured to apply a probe signal comprising a mechanical force arising from an electrostatic field or a magnetostatic field to said sample of interest.

9. An electromagnetic cellular tomography method, comprising the steps of:
providing an electromagnetic cellular tomograph, comprising:
an electromagnetic structure configured to apply a probe signal to a sample of interest and to receive a response signal from said sample of interest, said electromagnetic structure having at least one input terminal connected to an electromagnetic signal application structure and having at least one output terminal connected to an electromagnetic signal sensing structure;
a source circuit configured to provide an electromagnetic probe signal, said source circuit having at least one output terminal in electrical communication with said at least one input terminal of said electromagnetic structure configured to apply said probe signal to said at least one input terminal of said electromagnetic structure, and having at least one control input terminal configured to receive a control signal;
a sensing circuit configured to sense said response signal from said sample of interest, said sensing circuit having at least one input terminal to receive said response signal from said at least one output terminal of said electromagnetic structure, said sensing circuit having at least one output terminal at which said sensed response signal is provided;
a control circuit having at least one terminal in electrical communication with said at least one control input terminal of said source circuit, having at least one terminal in electrical communication with said at least one output terminal of said sensing circuit, and having at least one pair of input and output terminals for communication with an external electrical apparatus, said control circuit configured to control said source circuit, and said control circuit configured to communicate the sensed signals from the sensing circuit to said external electrical apparatus, and to accept control signals said external electrical apparatus; and
a sample holder adjacent said electromagnetic structure, said sample holder configured to hold a sample of interest;
providing a sample of interest in said sample holder;
applying a probe signal to said sample of interest;
sensing a response signal from said sample of interest;
analyzing said response signal to obtain a result; and
performing at least one of recording said result, transmitting said result to a data handling system, or to displaying said result to a user.

10. The electromagnetic cellular tomography method of claim 9, wherein said probe signal comprises an electrical current.

11. The electromagnetic cellular tomography method of claim 9, wherein said probe signal comprises an electrical voltage.

12. The electromagnetic cellular tomography method of claim 9, wherein said probe signal comprises an electrical field.

13. The electromagnetic cellular tomography method of claim 9, wherein said probe signal comprises a magnetic field.

14. The electromagnetic cellular tomography method of claim 9, wherein said probe signal comprises a mechanical force arising from an electrostatic field or a magnetostatic field.
